(19) United States
(12) Patent Application Publication
(10) Pub. No.: US 2009/0062571 A1
(43) Pub. Date: Mar. 5, 2009

(54) DEHYDRATION OF ALCOHOLS TO GIVE ALKENES OR ETHERS

(76) Inventors: Nikolai (Mykola) Ignatyev, Duisburg (DE); Urs Welz-Biermann, Heppenheim (DE); Karsten Koppe, Marl (DE); Peter Barthen, Rheinberg (DE); Hermann Josef Frohn, Kempen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/997,617

(22) PCT Filed: Jul. 5, 2006
(86) PCT No.: PCT/EP06/06554

§ 371 Date: Sep. 2, 2008

(30) Foreign Application Priority Data
Aug. 3, 2005 (DE) 10 2005 036 457.8

(51) Int. Cl. C07C 1/24; C07C 41/09; C09K 3/00

(52) U.S. Cl. 568/698; 585/639; 252/183.11

(57) ABSTRACT

The present invention relates to a process for the dehydration of alcohols, polyalcohols or alcoholates having at least one CH group in the α-position to the alcoholate or alcohol function to give alcenes or ethers, where the dehydration is carried out in ionic liquids of the general formula K⁺A⁻.

The present invention relates to a process for the dehydration of alcohols, polyalcohols or alcoholates having at least one CH group in the α-position to the alcoholate or alcohol function to give alkenes or ethers, where the dehydration is carried out in ionic liquids of the general formula K⁺A⁻.

The preparation of alkenes or ethers by elimination of water from alcoholates, in particular lithium or Grignard alcoholates, is known in principle and serves, for example, for the synthesis of aryl-substituted alkenes, which can be used, for example, as mesogenic substances, pharmaceutical active compounds, crop-protection agents, polymers or precursors in fine chemistry or for the preparation of corresponding starting compounds. For the elimination of water, the alcoholate which has a CH group in the r-position to the alcoholate function is usually reacted with an acid. The solvent used subsequently has to be removed by distillation or distilled off as an azeotrope together with the water formed. Alternatively, the dehydration can also be carried out heterogeneously using aluminium oxide as catalyst at temperatures of 300 to 400° C.

The above-mentioned processes have the disadvantage that either high temperatures have to be used, which may result in the decomposition of the organic compounds, or that large amounts of organic solvents have to be employed, which have to be removed again laboriously and disposed of correctly after the elimination of water has been carried out. For large-scale industrial syntheses in particular, the two methods mentioned thus prove to be disadvantageous since, in particular on use of solvents, additional safety aspects for avoiding environmental pollution and for fire protection have to be taken into account.

In order to avoid the use in solvents, WO 00/51957 proposes obtaining alkenes by heterogeneously acid-catalysed reaction of alcohols in supercritical solvents, such as, for example, supercritical CO₂₁ propane, halogenated hydrocarbons or nitrogen. However, the said use of supercritical solvents proves to be very laborious since the synthesis has to be carried out in corresponding pressure-tight containers and many of the said gases are either toxic or likewise flammable. The use of supercritical solvents is thus not very suitable for syntheses on commercial scales from safety and economic points of view.

The object of the present invention was therefore to provide a process of the type mentioned at the outset which enables the synthesis of the desired alkenes or of ethers in very high yields without the use of volatile solvents, that is readily controllable, does not require significant safety measures and thus also allows the economic synthesis of alkenes on commercial scales.

The above-mentioned object is achieved, surprisingly, by a process in accordance with the present invention. The present invention accordingly relates to a process for the dehydration of alcohols, polyalcohols or alcoholates having at least one CH group in the α-position to the alcoholate or alcohol function to give alkenes or ethers, where the dehydration is carried out in ionic liquids of the general formula K⁺A⁻. It has been found that ionic liquids are particularly suitable for carrying out the dehydration.

Ionic liquids or liquid salts are ionic species which consist of an organic cation (K⁺) and a generally inorganic anion (A⁻). They do not contain any neutral molecules and usually have melting points below 373 K.

Intensive research is currently being carried out in the area of ionic liquids since the potential applications are multifarious. Review articles on ionic liquids are, for example, R. Sheldon “Catalytic reactions in ionic liquids”, Chem. Commun., 2001, 2399-2407; M. J. Earle, K. R. Seddon “Ionic liquids. Green solvent for the future,” Pure Appl. Chem., 72 (2000), 1391-1398; P. Wasserscheid, W. Keim “Ionische Flüssigkeiten—neue Lösungen far die Übergangsmetalikatalyse” [Ionic Liquids—Novel Solutions for Transition-Metal Catalysis], Angew. Chem., 112 (2000), 3926-3945; T. Welton “Room temperature ionic liquids. Solvents for synthesis and catalysis”, Chem. Rev., 92 (1999), 2071-2083 or R. Hagiwara, Ya. Ito “Room temperature ionic liquids of alkylimidazolium cations and fluoroanions”, J. Fluorine Chem., 105 (2000), 221-227.

Since ionic liquids are salts, they have no volatility and thus also do not liberate any flammable or toxic vapours. They thus represent a safe medium for carrying out the dehydration reaction. In addition, it has been found that, on use of ionic liquids in the dehydration process, the addition of acid which is otherwise usual for catalysing the reaction is not absolutely necessary. The ionic liquid itself can thus catalyse the desired formation of alkenes or ethers. It has furthermore been found that the ionic liquids are capable of binding the water formed, enabling laborious separation of the water from the alkene or ether to be avoided. In the simplest case, the alkene formed or the ether can be decanted off from the ionic liquid and employed further without further purification. The ionic liquid too can be recycled simply in this manner and can be re-used a number of times. Overall, the process according to the invention proves to be a simple and inexpensive process which is also suitable for alkene or ether synthesis on a commercial scale.

Essential for the dehydration process according to the invention are the ionic liquids of the general formula K⁺A⁻. The choice of the anion A⁻ of the ionic liquid plays a particular role here. The anion A⁻ is preferably an anion of a corresponding strong acid. In particular, the anion A⁻ of the ionic liquid is selected from the group [HSO₄]⁻, [SO₄]⁻², [NO₃]⁻, [BF₄]⁻, [(R_{F})BF₃]⁻, [(R_{F})₂BF₂]⁻, [(R_{F})₃BF]⁻, [(R_{F})₄B]⁻, [B(CN)₄]⁻, [PO₄]⁻³, [HPO₄]²⁻, [H₂PO₄]⁻, [alkyl-OPO₃]⁻², [(alkyl-O)₂PO₂]⁻, [alkyl-PO₃]⁻, [R_{F}PO₃]⁻, [(alkyl)₂PO₂]⁻, [(R_{F})₂PO₂]⁻, [R_{F}SO₃]⁻, [alkyl-SO₃]⁻, [aryl-SO₃]⁻, [alkyl-OSO₃]⁻, [R_{F}C(O)O]⁻, [(R_{F}SO₂)₂N]⁻, {[(R_{F})₂P(O)]₂N}⁻, Cl⁻ and/or Br⁻,

where
R_{F} has the meaning fluorinated alkyl

(CₙF₂ₙ₋ₓ₊₁Hₓ)

where n=1-12 and x=0-7, where, for n=1, x should be =0 to 2, and/or fluorinated (also perfluorinated) aryl or alkylaryl.

The alkyl group in the above-mentioned anions can be selected from straight-chain or branched alkyl groups having 1 to 20 C atoms, preferably having 1 to 14 C atoms and particularly preferably having 1 to 4 C atoms.

R_{F} preferably denotes CF₃, C₂F₅, O₃F₇ or C₄F₉.

There are no restrictions per se with respect to the choice of the cation K⁺ of the ionic liquid. However, preference is given to organic cations, particularly preferably ammonium, phosphonium, thiouronium, guanidinium or heterocyclic cations.

Ammonium cations can be described, for example, by the formula (1)

[NR₄]⁺ (1),

where
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms, where one or more R may be partially or fully substituted by halogens, in particular —F and/or —Cl, or partially by —OR′, —CN, —C(O)OH, —C(O)NR′₂, —SO₂NR′₂, —C(O)X, —SO₂OH, —SO₂X or —NO₂, and where one or two non-adjacent carbon atoms of the R which are not in the α-position may be replaced by atoms and/or atom groups selected from the group —O—, —S—, —S(O)—, —SO₂—, —N⁺R′₂—, —C(O)NR′—, —SO₂NR′—, —P(O)(NR′₂)NR′—, or —P(O)R′—, where R′ may be ═H, non-, partially or perfluorinated C₁- to C₆-alkyl, C₃- to C₇-cycloalkyl, or unsubstituted or substituted phenyl, and X may be halogen.

Phosphonium cations can be described, for example, by the formula (2)

[PR²₄]⁺ (2),

where
R₂ in each case, independently of one another, denotes

### H, NR′₂,

straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms, where one or more R² may be partially or fully substituted by halogens, in particular —F and/or —Cl, or partially by —OR′, —CN, —C(O)OH, —C(O)NR′₂, —SO₂NR′₂, —C(O)X, —SO₂OH, —SO₂X or —NO₂, and where one or two non-adjacent carbon atoms of the R² which are not in the α-position may be replaced by atoms and/or atom groups selected from the group —O—, —S—, —S(O)—, —SO₂—, —N⁺R′₂—, —C(O)NR′—, —SO₂NR′—, —P(O)(NR′₂)NR′— or —P(O)R′—, where R′=H, non-, partially or perfluorinated C₁- to C₆-alkyl, C₃- to C₇-cycloalkyl, or unsubstituted or substituted phenyl, and X=halogen.

However, cations of the formulae (1) and (2) in which all four or three substituents R and R² are fully substituted by halogens are excluded, for example the tris(trifluoromethyl)methylammonium cation, the tetra(trifluoromethyl)ammonium cation or the tetra(nonafluorobutyl)ammonium cation.

Suitable thiouronium cations can be described by the formula (3)

[(R³R⁴N)—C(═SR⁵)(NR⁶R⁷)]⁺ (3)

where
R³ to R⁷ each, independently of one another, denote
hydrogen, where hydrogen is excluded for R⁵,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms, where one or more of the substituents R³ to R⁷ may be partially or fully substituted by halogens, in particular —F and/or —Cl, or partially by —OH, —OR′, —CON, —C(O)OH, —C(O)NR′₂, —SO₂NR′₂, —C(O)X, —SO₂OH, —SO₂X or —NO₂ and where one or two non-adjacent carbon atoms of R³ to R⁷ which are not in the α-position may be replaced by atoms and/or atom groups selected from the group —O—, —S—, —S(O)—, —SO₂—, —SO₂₀—, —C(O)O—, —N⁺R′₂—, —P(O)R′O—, —C(O)NR′—, —SO₂NR′—, —OP(O)R′O—, —P(O)(NR′₂)NR′—, —PR′₂═N— or —P(O)R′—, where R′=H, non-, partially or perfluorinated C₁- to C₆-alkyl, C₃- to C₇-cycloalkyl, or unsubstituted or substituted phenyl, and X=halogen.

Guanidinium cations can be described by the formula (4)

[C(NR⁸R⁹)(NR¹⁰R¹¹)(NR¹²R¹³)]⁺ (4),

where
R⁸ to R¹³ each, independently of one another, denote
hydrogen, —CN, NR′₂,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms, where one or more of the substituents R⁸ to R¹³ may be partially or fully substituted by halogens, in particular —F and/or —Cl, or partially by —OR′, —CN, —C(O)OH, —C(O)NR′₂, —SO₂NR′₂, —C(O)X, —SO₂OH, —SO₂X or —NO₂, and where one or two non-adjacent carbon atoms of R⁸ to R¹³ which are not in the α-position may be replaced by atoms and/or atom groups selected from the group —O—, —S—, —S(O)—, —SO₂—, —N⁺R)₂—, —C(O)NR′—, —SO₂NR′—, —P(O)(NR′₂)NR′— or —P(O)R′—, where R′=H, non-, partially or perfluorinated C₁- to C₆-alkyl, C₃- to C₇-cycloalkyl, or unsubstituted or substituted phenyl, and X=halogen.

In addition, it is possible to employ cations of the general formula (5)

[HetN]⁺ (5)

where
HetN⁺ denotes a heterocyclic cation selected from the group

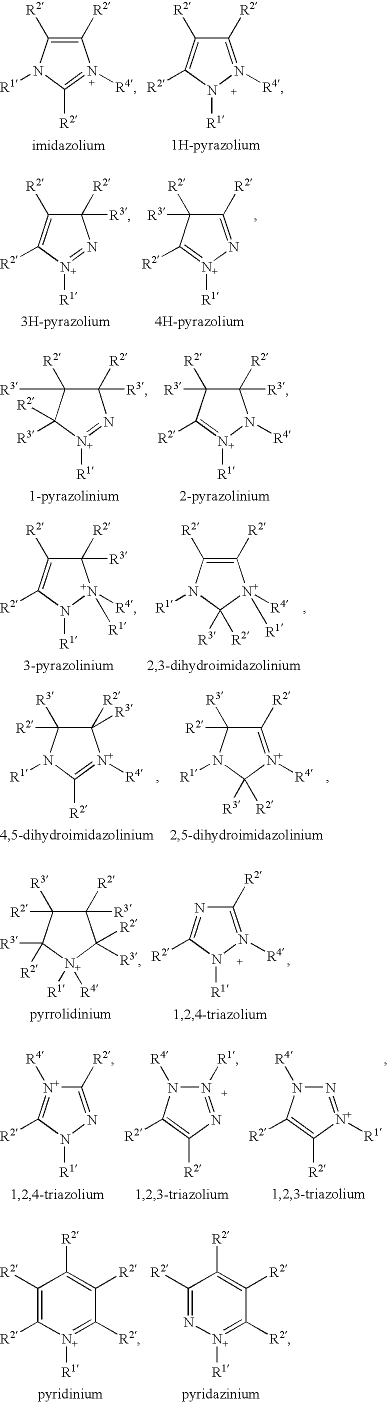

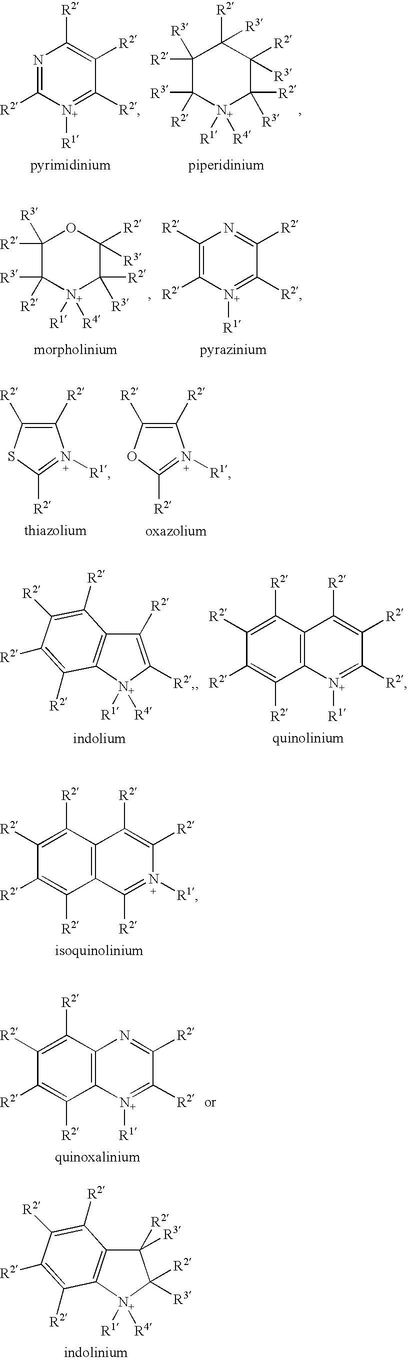

where the substituents
R^{1′} to R^{4′} each, independently of one another, denote hydrogen CN, —OR′, —NR′₂, —P(O)R′₂, —P(O)(NR′₂)₂, —C(O)R′,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
saturated, partially or fully unsaturated heteroaryl, heteroaryl-C₁-C₆-alkyl or aryl-C₁-C₆-alkyl,
where the substituents R^{1′}, R^{2′}, R^{3′} and/or R^{4′} together may also form a ring system,
where one or more substituents R^{1′} to R^{4′} may be partially or fully substituted by halogens, in particular-F and/or —Cl, or —OR′, —CN, —C(O)OH, —C(O)NR′₂, —SO₂NR′₂, —C(O)X, —SO₂OH, —SO₂X or —NO₂, but where R^{1′} and R^{4′} cannot simultaneously be fully substituted by halogens, and where one or two non-adjacent carbon atoms of the substituents R^{1′} to R^{4′} which are not bonded to the heteroatom may be replaced by atoms and/or atom groups selected from —O—, —S—, —S(O)—, —SO₂—, —C(O)—, —N⁺R′₂—, —C(O)NR′—, —SO₂NR′—, —P(O)(NR′₂)NR′—, —PR′₂—N— or —P(O)R′—, where R′=H, non-, partially or perfluorinated C₁- to C₆-alkyl, C₃- to C₇-cycloalkyl, or unsubstituted or substituted phenyl, and X=halogen.

For the purposes of the present invention, fully unsaturated substituents are also taken to mean aromatic substituents.

In accordance with the invention, suitable substituents R and R² to R¹³ of the compounds of the formulae (1) to (5), besides hydrogen, are preferably: C₁- to C₂₀-, in particular C₁- to C₁₄-alkyl groups, and saturated or unsaturated, i.e. also aromatic, C₃- to C₇-cycloalkyl groups, which may be substituted by C₁- to C₆-alkyl groups, in particular phenyl.

The substituents R and R² in the compounds of the formula (1) or (2) may be identical or different. The substituents R and R² are preferably different.

The substituents R and R² are particularly preferably methyl, ethyl, isopropyl, propyl, butyl, sec-butyl, tert-butyl, pentyl, hexyl, octyl, decyl or tetradecyl.

Up to four substituents of the guanidinium cation [C(NR⁸R⁹(NR¹⁰R¹¹)(NR¹²R¹³)]⁺ may also be bonded in pairs in such a way that mono-, bi- or polycyclic cations are formed.

Without restricting generality, examples of such guanidinium cations are:

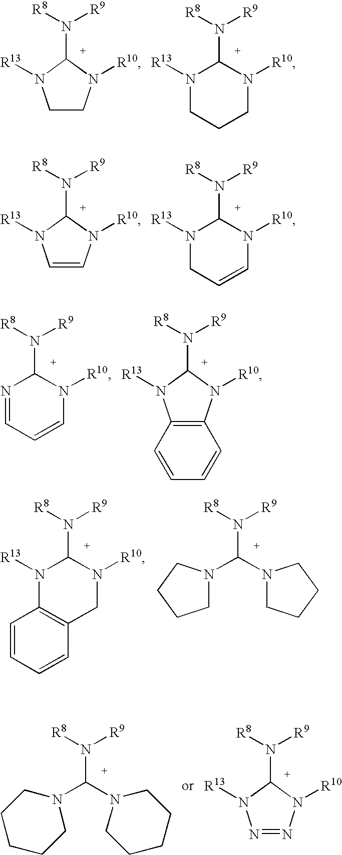

where the substituents R⁸ to R¹⁰ and R¹³ can have a meaning or particularly preferred meaning indicated above.

If desired, the carbocyclic or heterocyclic rings of the guanidinium cations indicated above may also be substituted by C₁- to C₆-alkyl, C₁- to C₆-alkenyl, NO₂, F, Cl, Br, I, OH, C₁-C₆-alkoxy, SCF₃₁ SO₂CF₃, COOH, SO₂NR′₂₁ SO₂X′ or SO₃H, where X and R′ have a meaning indicated above, substituted or unsubstituted phenyl or an unsubstituted or substituted heterocycle.

Up to four substituents of the thiouronium cation [(R³R⁴N)—C(═SR⁵)(NR⁶R⁷)]⁺ may also be bonded in pairs in such a way that mono-, bi- or polycyclic cations are formed.

Without restricting generality, examples of such cations are indicated below, where Y=S;

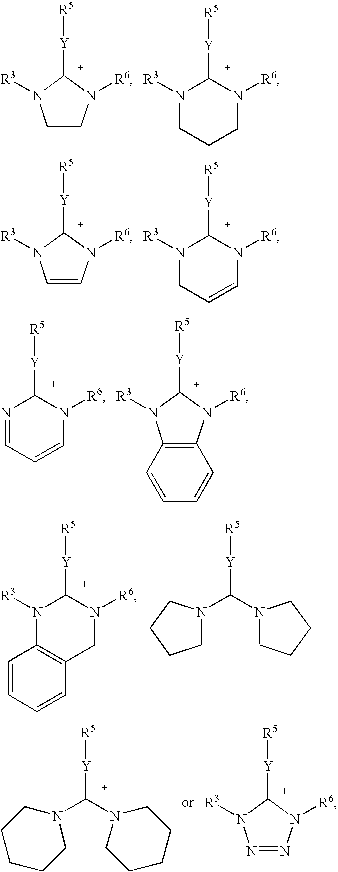

where the substituents R³, R⁵ and R⁶ can have a meaning or particularly preferred meaning indicated above.

If desired, the carbocyclic or heterocyclic rings of the cations indicated above may also be substituted by C₁- to C₆-alkyl, C₁- to C₆-alkenyl, NO₂, F, Cl, Br, I, C₁-C₆-alkoxy, SCF₃, SO₂CF₃, COOH, SO₂NR′₂, SO₂X or SO₃H or substituted or unsubstituted phenyl or an unsubstituted or substituted heterocycle, where X and R′ have a meaning indicated above.

The substituents R³ to R¹³ are each, independently of one another, preferably a straight-chain or branched alkyl group having 1 to 10 C atoms. The substituents R³ and R⁴, R⁶ and R⁷, R⁸ and R⁹, R¹⁰ and R¹¹ and R¹² and R¹³ in compounds of the formulae (3) to (5) may be identical or different here. R³ to R¹³ are particularly preferably each, independently of one another, methyl, ethyl, n-propyl, isopropyl, n-butyl, tert-butyl, sec-butyl, phenyl or cyclohexyl, very particularly preferably methyl, ethyl, n-propyl, isopropyl or n-butyl.

In accordance with the invention, suitable substituents R^{1′} to R^{4′} of compounds of the formula (5), besides hydrogen, are preferably: C₁- to C₂₀-, in particular C₁- to C₁₂-alkyl groups, and saturated or unsaturated, i.e. also aromatic, C₃- to C₇-cycloalkyl groups, which may be substituted by C₁- to C₆-alkyl groups, in particular phenyl.

The substituents R^{1′} and R^{4′} are each, independently of one another, particularly preferably methyl, ethyl, isopropyl, propyl, butyl, sec-butyl, tert-butyl, pentyl, hexyl, octyl, decyl, cyclohexyl, phenyl or benzyl. They are very particularly preferably methyl, ethyl, n-butyl or hexyl. In pyrrolidinium, piperidinium or indolinium compounds, the two substituents R^{1′} and R^{4′} are preferably different.

The substituent R^{2′} or R^{3′} is in each case, independently of one another, in particular hydrogen, methyl, ethyl, isopropyl, propyl, butyl, sec-butyl, tert-butyl, cyclohexyl, phenyl or benzyl. R^{2′} is particularly preferably hydrogen, methyl, ethyl, isopropyl, propyl, butyl or sec-butyl. R^{2′} and R^{3′} are very particularly preferably hydrogen.

The CO—C₁₋₂-alkyl group is, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl or dodecyl, optionally difluoromethyl, trifluoromethyl, pentafluoroethyl, heptafluoropropyl or nonafluorobutyl.

A straight-chain or branched alkenyl having 2 to 20 C atoms, in which a plurality of double bonds may also be present, is, for example, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉; preferably allyl, 2- or 3-butenyl, isobutenyl, sec-butenyl, furthermore preferably 4-pentenyl, isopentenyl or hexenyl.

A straight-chain or branched alkynyl having 2 to 20 C atoms, in which a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, preferably ethynyl, 1- or 2-propynyl, 2- or 3-butynyl, 4-pentynyl, 3-pentynyl or hexynyl.

Aryl-C₁-C₆-alkyl denotes, for example, benzyl, phenylethyl, phenylpropyl, phenylbutyl, phenylpentyl or phenylhexyl, where both the phenyl ring and also the alkylene chain may be partially or fully substituted, as described above, by halogens, in particular —F and/or —Cl, or partially by —OR′, —CN, —C(O)OH, —C(O)NR′₂, —SO₂NR′₂₁—C(O)X, —SO₂OH, —SO₂X, —NO₂.

Unsubstituted saturated or partially or fully unsaturated cycloalkyl groups having 3-7 C atoms are therefore cyclopropyl, cyclobutyl, cyclopentyl, cyclohexyl, cycloheptyl, cyclopentenyl, cyclopenta-1,3-dienyl, cyclohexenyl, cyclohexa-1,3-dienyl, cyclohexa-1,4-dienyl, phenyl, cycloheptenyl, cyclohepta-1,3-dienyl, cyclohepta-1,4-dienyl or cyclohepta-1,5-dienyl, each of which may be substituted by C₁- to C₆-alkyl groups, where the cycloalkyl group or the cycloalkyl group substituted by C₁- to C₆-alkyl groups may in turn also be substituted by halogen atoms, such as F, Cl, Br or I, in particular F or Cl, or by —OR′, —CN, —C(O)OH, —C(O)NR′₂, —SO₂NR′₂, —C(O)X, —SO₂OH, —SO₂X, —NO₂.

In the substituents R, R² to R¹³ or R^{1′} to R^{4′}, one or two non-adjacent carbon atoms which are not bonded in the α-position to the heteroatom may also be replaced by atoms and/or atom groups selected from the group —O—, —S—, —S(O)—, —SO₂—, —N⁺R′₂—, —C(O)NR′—, —SO₂NR′—, —P(O)(NR′₂)NR′— or —P(O)R′—, where R′=non-, partially or perfluorinated C₁- to C₆-alkyl, C₃- to C₇-cycloalkyl or unsubstituted or substituted phenyl.

Without restricting generality, examples of substituents R, R² to R¹³ and R^{1′} to R^{4′} modified in this way are:

—OCH₃, —OCH(CH₃)₂, —CH₂—OCH₃, —CH₂—CH₂—O—CH₃, —O₂H₄OCH(CH₃)₂, —C₂H₄C₂H₅, —C₂H₄SCH(CH₃)₂, —S(O)CH₃, —SO₂CH₃, —SO₂C₆H₅, —SO₂C₃H₇, —SO₂CH(CH₃)₂, —SO₂CH₂CF₃, CH₂SO₂CH₃, —O—C₄H₈—O—C₄H₉, —CF₃, —C₂F₅, —C₃F₇, —C₄F₉, —C(CF₃)₃, —CF₂SO₂CF₃, —C₂F₄N(C₂F₅)C₂F₅, —CHF₂, —CH₂CF₃, —C₂F₂H₃, —C₃FH₆, —CH₂C₃F₇, —C(CFH₂)₃, —CH₂C(O)OH, —CH₂C₆H₅ or P(O)(C₂H₅)₂.

In R′, C₃- to C₇-cycloalkyl is, for example, cyclopropyl, cyclobutyl, cyclopentyl, cyclohexyl or cycloheptyl.

In R′, substituted phenyl denotes phenyl which is substituted by C₁- to C₆-alkyl, C₁- to C₆-alkenyl, NO₂, F, Cl, Br, I, C₁-C₆-alkoxy, SCF₃, SO₂CF₃, COOH, SO₂X′, SO₂NR′₂ or SO₃H, where X′ denotes F, Cl or Br, and R″ denotes a non-, partially or perfluorinated C₁- to C₆-alkyl or C₃- to C₇-cycloalkyl as defined for R′, for example o-, m- or p-methylphenyl, o-, m- or pethylphenyl, o-, m- or p-propylphenyl, o-, m- or p-isopropylphenyl, o-, m- or p-tert-butylphenyl, o-, m- or p-nitrophenyl, o-, m- or p-methoxyphenyl, o-, m- or p-ethoxyphenyl, o-, m-, p-(trifluoromethyl)phenyl, o-, m-, p-(trifluoromethoxy)phenyl, o-, m-, p-(trifluoromethylsulfonyl)phenyl, o-, m- or p-fluorophenyl, o-, m- or p-chlorophenyl, o-, m- or p-bromophenyl, o-, m- or p-iodophenyl, furthermore preferably 2,3-, 2,4-, 2,5-, 2,6-, 3,4- or 3,5-dimethylphenyl, 2,3-, 2,4-, 2,5-, 2,6-, 3,4- or 3,5-dihydroxyphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

In R^{1′} to R^{4′}, heteroaryl is taken to mean a saturated or unsaturated mono- or bicyclic heterocyclic radical having 5 to 13 ring members, in which 1, 2 or 3 N and/or 1 or 2 S or O atoms may be present and the heterocyclic radical may be mono- or polysubstituted by C₁- to C₆-alkyl, C₁- to C₆-alkenyl, NO₂, F, Cl, Br, I, C₁-C₆-alkoxy, SCF₃, SO₂CF₃, COOH, SO₂X′, SO₂NR″₂ or SO₃H, where X′ and R″ have a meaning indicated above.

The heterocyclic radical is preferably substituted or unsubstituted 2- or 3-furyl, 2- or 3-thienyl, 1-, 2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -4- or -5-yl, 1- or 5-tetrazolyl, 1,2,3-oxadiazol-4- or -5-yl 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 4-4H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8-cinnolinyl, 2-, 4-, 5-, 6-, 7- or 8-quinazolinyl or 1-, 2- or 3-pyrrolidinyl.

Heteroaryl-C₁-C₆-alkyl is, analogously to aryl-C₁-C₆-alkyl, taken to mean, for example, pyridinylmethyl, pyridinylethyl, pyridinylpropyl, pyridinylbutyl, pyridinylpentyl or pyridinylhexyl, where the heterocycles described above may furthermore be linked to the alkylene chain in this way.

HetN⁺ is preferably

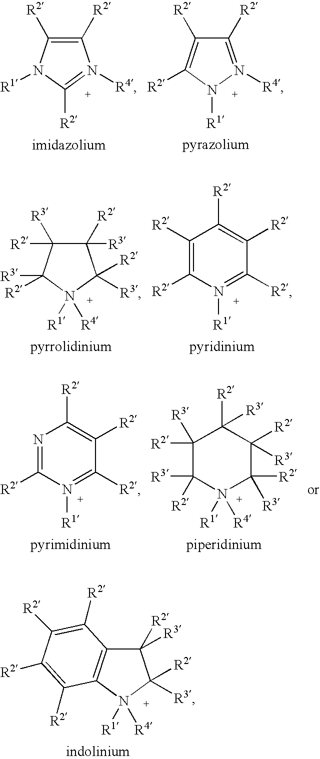

where the substituents R^{1′} to R^{4′} each, independently of one another, have a meaning described above.

The cations of the ionic liquid according to the invention are preferably ammonium, phosphonium, imidazolium, pyridinium or pyrrolidinium cations.

Particularly preferred ionic liquids are ammonium, phosphonium, imidazolium or pyrrolidinium hydrogensulfates, alkylsulfates, alkylsulfonates, perfluoroalkylsulfonates, phosphates, hydrogenphosphates, alkylphosphates, alkyl- and perfluoroalkylphosphinates, alkyl- and perfluoroalkylphosphonates or perfluoroalkylcarboxylates.

In a further preferred embodiment of the process according to the invention, the ionic liquid additionally comprises at least one acid, preferably an acid corresponding to the anion K. In general, any acid is suitable for mixing with the ionic liquid. Examples of preferred mixtures which prove to be particularly suitable in the processes according to the invention are, for example, mixtures of ionic liquids containing [HSO₄]⁻ anions and H₂SO₄. Alternative examples are mixtures of ionic liquids containing [CF₃SO₃]⁻ anions and CF₃SO₃H or mixtures of ionic liquids containing [CF₃C(O)O]⁻ anions and CF₃C(O)OH. The said mixtures should be regarded as illustrative here without representing a limitation of the possibilities of the present invention.

The proportion of the acid in the ionic liquid can be 0 to 90% by weight, based on the mixture, preferably in the range from 0 to 50% by weight.

The process temperature is not crucial per se and is usually 0 to 170° C., preferably 20 to 120° C.

The said mixtures of ionic liquids and at least one acid are particularly suitable in the processes according to the invention since the dehydration reaction proceeds more quickly than with the ionic liquid alone. In addition, it has been found that, in particular, mixtures of ionic liquids and acids corresponding to the anion A⁻ of the ionic liquid are distinguished by the fact that the acid has low volatility in the mixture, i.e. is present in the mixture in constant concentration, even at elevated temperatures. Thus, for example, trifluoroacetic acid proves to be virtually non-volatile and has only a low vapour pressure in the mixture with an ionic liquid containing a trifluoroacetate anion.

Overall, water eliminations from alcoholates or alcohols or polyalcohols which could not be carried out by means of known processes are therefore accessible by means of the novel process according to the invention, and at the same time the dehydration reactions can be optimised significantly better. In the system described, the elimination of water from alcohols is possible in two ways; intramolecularly or intermolecularly. In the first case, alkenes form, while in the second case, ethers, for example dialkyl ethers, result. The process according to the invention is preferably used for the preparation of alkenes.

The processes according to the invention are suitable for the elimination of water not only from alcohols, but also from polyalcohols, for example glycols, triols, or natural products, such as, for example, polysaccharides, hexoses or pentoses.

The process according to the invention is particularly advantageously employed for the synthesis of aryl-substituted alkenes, which are used, for example, as mesogenic substances, pharmaceutical active compounds, crop-protection agents, polymers or precursors in fine chemistry or for the preparation of corresponding starting compounds.

The alcoholate or alcohol used, jointly referred to as alcoholate below, is preferably a compound of the formula I

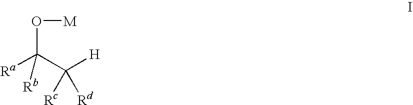

in which
M denotes an alkali metal, an alkaline-earth metal halide or an H atom, and
R^{a}, R^{b}, R^{c}, R^{d}, independently of one another, denote an optionally substituted aliphatic or aromatic radical, which may have one or more hetero atoms, where one, two or three radicals from the group R^{a} to R^{d} may also denote H, and where two or more of the radicals R^{a}, R^{b}, R^{c} and/or R^{d} may be connected to one another.

The radicals R^{a} and R^{b} and/or R^{b} and R^{c} are preferably connected to one another, for example with formation of an aliphatic and/or aromatic ring or fused ring system, which may also have one or more hetero atoms.

Preferred meanings of M are H, Li, MgCl, MgBr or MgI.

R^{a} preferably has a meaning of the formula Ia

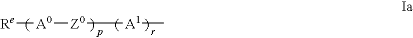

in which
R^{e} denotes an alkyl radical having 1 to 15 C atoms which is unsubstituted or mono- or polysubstituted by CN and/or halogen, where, in addition, one or more CH₂ groups in these radicals may be replaced by —O—, —S—, —CH≡CH—, —C═C—, —O—O— and/or —O—CO— and/or, in addition, one or more CH groups may be replaced by N or P in such a way that two O atoms are not linked directly to one another, or, if r and/or p are not 0, also H, halogen, CN, SF₅ or NCS,
A⁰, A¹ each, independently of one another, denote
   a) a 1,4-cyclohexenylene or 1,4-cyclohexylene radical, in which one or two non-adjacent CH₂ groups may be replaced by —O— or —S—,
   b) a 1,4-phenylene radical, in which one or two CH groups may be replaced by N,
   c) a radical from the group piperidine-1,4-diyl, 1,4-bicyclo-[2.2.2]octylene, phenanthrene-2,7-diyl, naphthalene-2,6-diyl, decahydronaphthalene-2,6-diyl and 1,2,3,4-tetrahydronaphthalene-2,6-diyl,
   d) a divalent radical from the group furan, pyrrole, thiophene, pyrazole, imidazole, 1,2-oxazole, 1,3-oxazole, thiazole, pyridine, pyridazine, pyrimidine, pyrazine, 2H-pyran, 4H-pyran, purine, pteridine, 1H-azepine, 3H-1,4-diazepine, indole, benzofuran, benzothiophene, quinoline, isoquinoline, phenazine, phenoxazine, phenothiazine and 1,4-benzodiazepine,
   where the radicals a), b), c) and d) may be mono- or polysubstituted by R^{e}, in particular by halogen and/or CN,
Z⁰ denotes —CO—O—, —O—CO—, —CF₂O—, —OCF₂—, —CH₂O—, —OCH₂—, —CH₂CH₂—, —(CH₂)₄—, —C₂F₄—, —CH₂CF₂—, —CF₂CH₂—, —CF═CF—, —CH═C H—, —C≡C— or a single bond,
p denotes 0, 1, 2 or 35 and
r denotes 0, 1 or 2.

Preferred meanings of R^{e} are straight-chain or branched alkyl and alkoxy radicals having 1 to 8 C atoms, which may be monosubstituted by —CN and/or mono- or polysubstituted by halogen.

Preferred meanings of A⁰ and/or A¹ are 1,4-cyclohexylene, in which one or two non-adjacent CH₂ groups may be replaced by —O—, 1,4-phenylene, in which one or two CH groups may be replaced by N, phenanthrene-2,7-diyl, naphthalene-2,6-diyl, decahydronaphthalene-2,6-diyl and 1,2,3,4-tetrahydronaphthalene-2,6-diyl, where these radicals may be mono- or polysubstituted by halogen, in particular fluorine and/or chlorine, CN and/or C₁₋₅-alkyl or -alkoxy which is optionally substituted by halogen.

A¹ is particularly preferably a 1,4-phenylene group, which is unsubstituted or mono-, di-, tri- or tetrasubstituted by fluorine in the 2-, 3-, 5- and/or 6-position, whereby the reaction according to the invention proceeds in accordance with the following scheme:

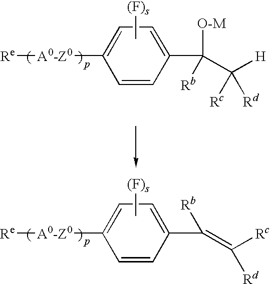

in which R^{e}, A⁰, Z⁰, p, M, R^{b}, R^{c} and R^{d} have the meanings indicated above and below, and s denotes 0, 1, 2, 3 or 4.

Preferably:
p=r=0, where R′ is preferably a straight-chain or branched alkyl radical having 1 to 8 C atoms, which may be monosubstituted by —CN and/or mono- or polysubstituted by halogen,
r=1 and p=0, 1 or 2.

R^{a} may also be a constituent of a ligand, for example of a cyclopentadienyl system in an organometallic complex.

Particularly preferred groups R^{a} are shown below:

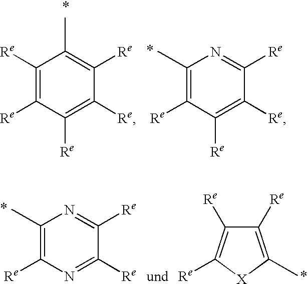

in which X denotes Q, NR^{e} or S, R^{e} has the meaning indicated, and * indicates the free bond.

Above and below, halogen as substituent of organic radicals denotes fluorine, chlorine, bromine or iodine, preferably fluorine or chlorine, particularly preferably fluorine.

Above and below, groups and substituents which occur more than once, such as, for example, A⁰, Z⁰, A¹, R^{e}, may each have identical or different meanings.

Preferably, one, two or three radicals from the group R^{b}, R^{c}, R^{d} have, independently of one another, a meaning of the formula Ia, and any other radicals from the group R^{b}, R^{c}, R^{d} denote H.

Particularly preferably, R^{d} is H, and R^{b} and/or R^{c} have a meaning other than H. R^{b} and R^{c} are very particularly preferably different from H.

R^{b} and R^{c} are furthermore preferably connected to one another in such a way that the alcoholate of the formula I has a meaning of the formula Ib

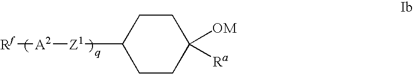

in which
R^{f} has one of the meanings indicated for R^{e},
A² has one of the meanings indicated for A⁰, A¹,
Z¹ has one of the meanings indicated for Z⁰,
q denotes 0, 1, 2 or 3, and

R^{a} and M have the meanings indicated above and below.

The alcoholates of the formula I are obtainable in good to very good yields by the addition reaction of organometallic compounds onto compounds having one or more carbonyl functions. Reactions of this type and the starting materials, solvents and reaction conditions to be employed are known to the person skilled in the art or can readily be obtained by modification of known syntheses.

It goes without saying to the person skilled in the art that substituents such as, for example, H, N, O, Cl, F in the said ionic liquids or alcohols or alcoholates may be replaced by the corresponding isotopes.

The present invention likewise relates to mixtures of ionic liquids of the general formula K⁺A⁻ and at least one acid. The at least one acid is preferably an acid corresponding to the anion A⁻ of the ionic liquid. These said mixtures allow dehydration reactions to be carried out with a multiplicity of substrates. In addition, preferred mixtures of ionic liquids of the general formula K⁺A⁻ with acids corresponding to the anion A⁻ are characterised in that the acid has low volatility in the mixture, and a constant acid concentration can thus be achieved more easily.

The mixtures of ionic liquids and acids corresponding to the anion A⁻ thus represent a novel class of strongly acidic systems of low volatility of free acid. The said mixtures can be used as replacement for volatile organic and inorganic acids in various applications, for example as component of etching agents (pastes), as catalysts in various processes, for example in Friedel-Crafts alkylations or acylations or in alkane isomerisations, or as components of electrolytes for electrochemical cells. The present invention thus likewise relates to the use of mixtures of ionic liquid and acid as replacement for volatile organic and inorganic acids in various applications.

The proportion of the at least one acid in the mixtures according to the invention is in the above-mentioned ranges.

The following working examples are intended to explain the invention without limiting it. Above and below, percentage data denote percent by weight. All temperatures are indicated in degrees Celsius.

### EXAMPLES

### Example 1

### Synthesis of 1-phenylcyclohex-1-ene

1-Phenyl-1-cyclohexanol is added to 10 ml of ethylmethylimidazolium hydrogensulfate, and the mixture is stirred at 80-90° C. for one hour. After cooling, two phases form, with the upper phase, the product phase, being decanted off. 1-Phenyl-1-cyclohexanol is again added to the lower phase, the ionic liquid, which is correspondingly reacted and separated off. The said procedure can be repeated a number of times without changing the ionic liquid. The average yield of 1-phenylcyclohex-1-ene is 97.2%, the product can be purified further by distillation.

The isolated product is analysed by means of NMR spectroscopy.

¹H NMR (reference: TMS; solvent: CD₃CN), ppm: 1.90 m (CH₂); 2.01 m, (CH₂); 2.43 m (CH₂); 2.64 m (CH₂); 6.36 m (1CH); 7.54 m (5CH, Ph).

### Example 2

### Synthesis of Isobutylene

Tert-butanol is added to a mixture of ethylmethylimidazolium hydrogensulfate and concentrated sulfuric acid (volume ratio 3.75:1). The reaction mixture (an emulsion) is stirred at 43° C. for 4 hours. Isobutylene formed is condensed in a trap at −196° C. (liquid nitrogen) and atmospheric pressure. The trap is subsequently warmed to −78° C., melted and weighed at room temperature. Isobutylene is isolated as a clear and colourless liquid. The said procedure can be repeated a number of times without changing the ionic liquid.

The average yield of isolated isobutylene is 92%.

The isolated product is analysed by means of NMR spectroscopy.

¹H NMR (reference: TMS, solvent: CDCl₃), ppm; 1.55 t (2CH₃); 4.49 sep, (CH₂); ⁴J_{H,H}=1.1 Hz.

### Example 3

### Synthesis of Cyclohexene

Concentrated sulfuric acid (97-98%) is added to a mixture of ethylmethylimidazolium hydrogensulfate and cyclohexanol (volume ratio 1:1.7:2). After a highly exothermic reaction and vigorous stirring, the emulsion homogenises. The solution formed is stirred at 75° C. for one hour, and cyclohexene formed is distilled off. The yield of cyclohexene is 82%.

The isolated product is analysed by means of NMR spectroscopy.

¹H NMR (reference: TMS; solvent: CDCl₃), ppm: 1.50 m (2CH₂); 1.87 ml (2CH₂); 5.53 m (20H).

### Example 4

### Synthesis of 2,3-dimethylbuta-1,3-diene

A mixture of ethylmethylimidazolium hydrogensulfate and 2,3-dimethyl-2,3-butanediol (weight ratio 1:1) is heated to 140° C., and 2,3-dimethylbuta-1,3-diene formed is distilled off under atmospheric pressure together with other dehydration products (such as, for example, 2,3-epoxy-2,3-dimethylbutane). The pure 2,3-dimethylbuta-1,3-diene can be isolated by subsequent fractional distillation. The yield of isolated 2,3-dimethylbuta-1,3-diene is 60%. The said procedure can be repeated a number of times without changing the ionic liquid.

The isolated product is analysed by means of NMR spectroscopy.

¹H NMR (reference: TMS; solvent: CDCl₃), ppm: 1.68 s (2CH₃); 4.73 m, (2CH); 4.82 m (2CH).

### Example 5

### Synthesis of Diheptyl Ether

Concentrated sulfuric acid (97-98%) is added to a mixture of ethylmethylimidazolium hydrogensulfate and 1-heptanol (volume ratio 1:2:1.3). After an exothermic reaction and vigorous stirring, the emulsion homogenises. The solution formed is stirred at 117° C. for 2.5 hours, and diheptyl ether formed is extracted and isolated by fractional distillation. The yield of isolated diheptyl ether is 50%.

The isolated product is analysed by means of NMR spectroscopy.

¹H NMR (reference: TMS; solvent: CD₃CN), ppm: 0.85 m (2CH₂); 1.27 m, (8CH₂); 1.30 m (2CH₂); 3.70 t (2CH₂); ³J_{H,H}=6.8 Hz. ¹³C {¹H} NMR (reference: TMS; solvent: CD₃CN), ppm: 14.1 s; 23.0 s; 25.9 s; 29.1 s; 29.4 s; 32.0 s; 70.1 s.

### Example 6

### Mixtures of 1-butyl-3-methylimidazolium trifluoroacetate with trifluoroacetic acid

10% by weight (or 20% by weight) of trifluoroacetic acid are added to 1-butyl-3-methylimidazolium trifluoroacetate. The resultant mixture is analysed by the TGA method.

The mixture with 20% by weight of trifluoroacetic acid (boiling point of free acid is 72-73° C.) has a weight loss of only about 2.5% at 140° C.

The mixture with 10% by weight of trifluoroacetic acid has a weight loss of only less than 2% at 140° C.

1. Process for the dehydration of alcohols, polyalcohols or alcoholates having at least one CH group in the α-position to the alcoholate or alcohol function to give alkenes or ethers, characterised in that the dehydration is carried out in ionic liquids of the general formula K⁺A⁻.

2. Process according to claim 1, characterised in that the anion A⁻ of the ionic liquid is selected from the group [HSO₄]⁻, [SO₄]⁻², [NO₃]⁻, [BF₄]⁻, [(R_{F})BF₃]⁻, [(R_{F})₂BF₂]⁻, [(R_{F})₃BF]⁻, [(R_{F})₄B]⁻, [B(CN)₄]⁻, [PO₄]⁻³, [HPO₄]⁻², [H₂PO₄]⁻, [alkyl-OPO₃]⁻², [(alkyl-O)₂PO₂]⁻, [alkyl-PO₃]⁻, [R_{F}PO₃]⁻, [(alkyl)₂PO₂]⁻, [(R_{F})₂PO₂]⁻, [R_{F}SO₃]⁻, [alkyl-SO₃]⁻, [aryl-SO₃]⁻, [alkyl-OSO₃]⁻, [R_{F}C(O)O]⁻, [(R_{F}SO₂)₂N]⁻, {[(R_{F})₂P(O)]₂N}⁻, Cl⁻ and/or Br⁻, where R_{F} has the meaning fluorinated alkyl
(CₙF₂ₙ₋ₓ₊₁Hₓ)
where n=1-12 and x=0-7, where, for n=1, x should be =0 to 2, and/or fluorinated aryl or alkylaryl.

3. Process according to claim 1, characterised in that the cations K⁺ of the ionic liquid are selected from the group of the ammonium, phosphonium, thiouronium, guanidinium and heterocyclic cations.

4. Process according to claim 1, characterised in that the ionic liquid additionally comprises an acid corresponding to the anion A⁻.

5. Process according to claim 4, characterised in that the proportion of the acid in the ionic liquid is 0 to 90% by weight, based on the mixture.

6. Process according to claim 1, characterised in that the process temperature is 0 to 170° C.

7. Process according to claim 1, characterised in that the alcohol is selected from the compounds of the formula (I) 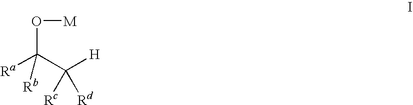
in which
M denotes an alkali metal, an alkaline-earth metal halide or an H atom, and
R^{a}, R^{b}, R^{c}, R^{d}, independently of one another, denote an optionally substituted aliphatic or aromatic radical, which may have one or more hetero atoms, where one, two or three radicals from the group R^{a} to R^{d} may also denote H, and where two or more of the radicals R^{a}, R^{b}, R^{c} and/or R^{d} may be connected to one another.

8. Process according to claim 7, characterised in that M denotes H, Li, MgCl, MgBr or MgI.

9. Process according to claim 7, characterised in that R^{a} has a meaning of the formula Ia 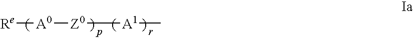
in which
R^{e} denotes an alkyl radical having 1 to 15 C atoms which is unsubstituted or mono- or polysubstituted by CN and/or halogen, where, in addition, one or more CH₂ groups in these radicals may be replaced by —O—, —S—, —CH═CH—, —C≡C—, —OC—O— and/or —O—CO— and/or, in addition, one or more CH groups may be replaced by N or P in such a way that two O atoms are not linked directly to one another, or, if r and/or p are not 0, also H, halogen, CN, SF₅ or NCS,
A⁰, A¹ each, independently of one another, denote
a) a 1,4-cyclohexenylene or 1,4-cyclohexylene radical, in which one or two non-adjacent CH₂ groups may be replaced by —O— or —S—,
b) a 1,4-phenylene radical, in which one or two CH groups may be replaced by N,
c) a radical from the group piperidine-1,4-diyl, 1,4-bicyclo-[2.2.2]octylene, phenanthrene-2,7-diyl, naphthalene-2,6-diyl, decahydronaphthalene-2,6-diyl, and 1,2,3,4-tetrahydronaphthalene-2,6-diyl,
d) a divalent radical from the group furan, pyrrole, thiophene, pyrazole, imidazole, 1,2-oxazole, 1,3-oxazole, thiazole, pyridine, pyridazine, pyrimidine, pyrazine, 2H-pyrane, 4H-pyran, purine, pteridine, 1H-azepine, 3H-1,4-diazepine, indole, benzofuran, benzothiophene, quinoline, isoquinoline, phenazine, phenoxazine, phenothiazine and 1,4-benzodiazepine,
where the radicals a), b), c) and d) may be mono- or polysubstituted by R^{e}, in particular by halogen and/or CN,
Z⁰ denotes —CO—O—, —O—CO—, —CF₂O—, —OCF₂—, —CH₂O—, —OCH₂—, —CH₂CH₂—, —(CH₂)₄—, —C₂F₄—, —CH₂CF₂—, —CF₂CH₂—, —CF═CF—, —CH═CH—, —C≡C— or a single bond,
p denotes 0, 1, 2 or 3, and
r denotes 0, 1 or 2.

10. Process according to claim 9,
characterised in that one, two or three radicals from the group R^{b}, R^{c}, R^{d} have, independently of one another, a meaning of the formula Ia according to claim 9, and any other radicals from the group R^{b}, R^{c}, R^{d} denote H.

11. Mixtures of ionic liquids of the general formula K⁺A⁻ and at least one acid.

12. Mixtures according to claim 11, characterised in that the acid is an acid corresponding to the anion A⁻ of the ionic liquid.

13. Use of mixtures according to claim 11 as replacement for volatile organic and inorganic acids.
